(19) United States
(12) Patent Application Publication
(10) Pub. No.: US 2015/0208923 A1
(43) Pub. Date: Jul. 30, 2015

(54) Non-Invasive Monitoring of Tissue Mechanical Properties

(71) Applicants: The Texas A&M University System, College Station, TX (US); Oak Ridge National Laboratory, Oak Ridge, TN (US); University of Pittsburgh - Of the Commonwealth System of Higher Education, Pittsburgh, PA (US); The United States Government, Washington, DC (US)

(72) Inventors: Tony J. Akl, College Station, TX (US); Gerard L. Coté, College Station, TX (US); Mark A. Wilson, Sewickley, PA (US); Milton Nance Ericson, Knoxville, TN (US); John P. Hanks, College Station, TX (US)

(21) Appl. No.: 14/608,185

(22) Filed: Jan. 28, 2015

Related U.S. Application Data
(60) Provisional application No. 61/932,575, filed on Jan. 28, 2014.
(60) Provisional application No. 61/932,567, filed on Jan. 28, 2014.

(51) Int. Cl. A61B 5/00; A61B 5/02

(52) U.S. Cl. CPC A61B 5/0084; A61B 5/02007; A61B 5/4878; A61B 5/6847

(57) ABSTRACT

Methods and apparatuses for a tissue mechanical property monitoring system are disclosed herein. In one embodiment, a tissue mechanical property monitoring system is disclosed. The tissue mechanical property monitoring system may comprise a probe, wherein the probe comprises a light source and a photodetector; and a main unit, wherein the main unit comprises a microcontroller and wireless transmitter. The probe may be hermetically sealed and may be capable of being implanted onto tissue. The photodetector may be capable of collecting reflectance data from the light emitted by the light source. The reflectance data may be capable of being sorted and processed into tissue mechanical property data such as tissue compliance, vascular resistance, and the like for the tissue illuminated with the probe.

### CROSS-REFERENCE TO RELATED APPLICATIONS

This application claims benefit of U.S. Provisional Application Ser. Nos. 61/932,575 entitled “Non-Invasive Monitoring of Tissue Mechanical Properties” and 61/932,567 entitled “Arterial and Venous Oxygenation Method and Apparatus”, both of which were filed Jan. 28, 2014 and are incorporated herein by reference in their entirety.

This application is also related to U.S. patent application Ser. No. 14/608,145 filed currently herewith and entitled “Arterial and Venous Oxygenation Method and Apparatus”, which is incorporated herein by reference in its entirety.

### STATEMENT REGARDING FEDERALLY SPONSORED RESEARCH OR DEVELOPMENT

This invention was made with government support under 5R01-GM077150 awarded by National Institutes of Health (NIH). The government has certain rights in the invention.

### BACKGROUND OF THE INVENTION

1. Field of the Invention

The present embodiments relate to measuring tissue mechanical properties in vivo, and more specifically to measuring the tissue mechanical properties to detect developing problems within the tissue or to ascertain the state of the tissue.

2. Background of the Invention

The mechanical properties of tissue may be affected by many disease and injuries such as fibrosis and/or burns. Traditionally, measuring the mechanical properties of tissues can be invasive, most sampling occurs by taking biopsies of specific tissue areas. Non-invasive technologies have been developed but they do not provide enough resolution to distinguish between disease states. As such, these techniques may be too insensitive a measure for a clinician to provide timely intervention in the instance of a developing problem.

As an example, current non-invasive technologies such as magnetic resonance imaging (MRI), may only distinguish between normal (F0) and cirrhotic (F4) liver tissue. MRI is unable to ascertain a distinction between any of the other stages of liver disease, such that the early detection of fibrosis is difficult. Likewise, less sensitive monitoring techniques may also hinder the ability of physicians or other healthcare providers to study the progress of disease/healing of tissue.

Photoplethysmography (PPG) is a commonly used noninvasive method to record a pulse. However, the waveform of the pulse is typically ignored while the frequency and amplitude of the pulses are used instead to provide diagnostic information. The pulse waveform may carry substantial information that can itself be used to provide information about tissue mechanical properties. The pulse waveform can be used by itself or in conjunction with other diagnostics to provide information regarding fibrosis, cirrhosis, wound healing, tissue burn monitoring, edema, and many other conditions.

Consequently, there is a need for a more sensitive quantification of tissue mechanical properties.

### BRIEF SUMMARY OF SOME OF THE PREFERRED EMBODIMENTS

These and other needs in the art are addressed in one embodiment by a tissue mechanical property monitoring system comprising a probe and a main unit. The probe further comprises light sources and one or more photodetectors. The main unit drives the light sources, collects the data from the detectors, and then processes and displays the measurements. In some embodiments the main unit may transmit the data wirelessly to a processing and/or monitoring unit which may comprise a personal computing device (e.g., computer, smart-phone, tablet, and the like).

An additional embodiment comprises a method for measuring tissue mechanical properties in vivo using light sources, photodetectors, and data collection/manipulation. The method may comprise exposing tissue to light at different wavelengths generated by light sources such as light emitting diodes, measuring the reflectance of the light via a photodetector to produce a reflectance signal. Analyzing and manipulating the reflectance signal such that the differences in the tissue or interest are isolated from corresponding readings of peripheral tissue. Optionally, the method may further comprise reducing the measurements to a display relating the signal data to information regarding the tissue's mechanical properties.

Another embodiment includes a probe and a main unit. The probe includes a light source and a photodetector. The main unit includes a microcontroller and a communications interface with the probe. The photodetector is configured to collect a reflectance data from a light emitted by the light source that illuminates a tissue. The microcontroller processes the reflectance data into a tissue mechanical property date for the tissue. An additional probe communicably coupled to the one or more processors and configured to measure peripheral readings for the tissue may also be provided.

Yet another embodiment includes a method for monitoring mechanical properties of a tissue. A probe is provided that is affixed to or in close proximity to a surface of the tissue. The probe includes one or more light sources and one or more photodetectors. One or more processors communicably coupled to the probe and a data output device are also provided. The tissue is illuminated using the one or more light sources, and a reflectance signal is detected using the one or more photodetectors. The mechanical properties for the tissue are determined based on the reflectance signal using the one or more processors. The mechanical properties are then provided to the output device. An additional probe communicably coupled to the one or more processors and configured to measure peripheral readings for the tissue may also be provided.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other embodiments for carrying out the same purposes of the present invention. It should also be realized by those skilled in the art that such equivalent embodiments do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates a flowchart of the process;

FIG. 2 illustrates a flowchart of the process using two sensors;

FIG. 3A illustrates a schematic of the PPG signal showing the AC and DC signals;

FIG. 3B illustrates changes in the shape of the pulse with arterial compliance;

FIG. 4 illustrates a PPG waveform obtained by a Windkessel model showing the detected peaks (circles) and valleys (x); the green symbols show the 10% threshold;

FIG. 5 illustrates the mechanical properties of two different PDMS phantoms with different curing parameters;

FIG. 6 illustrates a schematic of the tissue mechanical property monitoring system and the flow system used to test it;

FIG. 7 illustrates a schematic of the four-element Windkessel model used to simulate the arterial pulse;

FIG. 8A illustrates modeled blood flow;

FIG. 8B illustrates three waveforms with different mechanical properties showing the changes in the pulse shape;

FIG. 9A illustrates a pulse measured from a soft (15 KPa) phantom;

FIG. 9B illustrates a pulse measured from a stiff (61 KPa) phantom;

FIG. 10 illustrates changes in the pulse rise time with compliance;

FIGS. 11A and 11B illustrate an example of a downstream (FIG. 11A) and an upstream occlusion (FIG. 11B) in which the amplitude of the pulse (top line) decreases indicating a drop in flow level, and the rise time (bottom line) increased only in the case of downstream occlusions;

FIG. 12 illustrates a bar plot of the change in rise time during upstream (USO) and downstream (DSO) occlusions;

FIG. 13A illustrates changes in the rise time and fall time of the PPG pulse for different compliance values simulated using the Windkessel model, and FIG. 13B shows the data after conversion of the compliance values to YM; and

FIG. 14 illustrates changes in the pulse rise and fall time for different levels of vascular resistance.

### DETAILED DESCRIPTION OF THE PREFERRED EMBODIMENTS

Embodiments may comprise one or more probes. The probes may be placed at a specific application and measurement site. The probes may be composed of any material sufficient for contact with the internal or external structures of a living organism. Such materials may be defined as biocompatible materials. Examples of general materials include, but should not be limited to metals, plastics, and the like. Without limitation, specific examples of materials may include polyethylene glycol, poly(methyl methacrylate), polydimethylsiloxane, parylene, titanium, combinations and composites thereof, and the like. Alternatively, the probes may be encapsulated using any of the above mentioned materials such that any portion of the probes (e.g., the electronic portions of the probes) is hermetically sealed and/or moisture tight.

Embodiments of the probes may further comprise one or more light sources. Multiple light sources may be used for a given application, including a combination of different models or types of light sources. The light source may be any light source sufficient for measuring the levels of tissue oxygen metabolism. Examples include light emitting diodes (LEDs), lasers, etc. The light source may produce light of any wavelength. Light sources that produce wavelengths of light in the near infrared range penetrate deeper in the tissue and may carry higher oxygenation signal levels and thus may be preferred for some applications. Examples of potential light wavelengths include 735, 805, and 940 nm. Optionally, the LEDs may be time multiplexed or frequency multiplexed such that only a single photodetector may be needed to collect the diffuse reflectance at each of the wavelengths. Moreover, the light produced from the light source may be modulated such that the produced light is at a different frequency than the ambient light and may therefore be distinguished from any ambient light noise. Modulation may comprise frequency modulation, time division multiplexing, or a combination of the two. Any technique for modulation that allows the light source to produce a light at a different frequency than the ambient light of the surrounding system may be sufficient for applications.

Embodiments of the probe may further comprise one or more photodetectors. In embodiments where multiple photodetectors are used, the photodetectors may be set up as an array. The photodetector may be any photodetector sufficient for measuring the light reflectance of the light source. Without limitation, the photodetectors may comprise solid state photodetectors, specific examples of which may include silicon photodetector, photo multiplier tubes, charge-coupled devices (CCD), avalanche photodiodes, electron-multiplying charge-coupled device, and the like. Certain types of photodetectors, such as CCD photodetectors may comprise cameras. Multiple photodetectors may be used for a given application, including a combination of different models or types of photodetectors. The photodetector should be sensitive to the wavelength of light produced by the light source. In some embodiments a single photodetector may measure the reflected light from multiple light sources. The photo detector may be composed of any material sufficient for measuring reflected light and potentially also sufficient for contact with the internal structures of a living organism. Examples of materials include metals, plastics, and the like.

In embodiments, the probes may be used invasively or noninvasively. For example, the probes may be implantable such that is affixed to the tissue or organ either on the surface of the tissue or organ or subcutaneously inserted into the tissue or the organ. The probes may be affixed to or inserted into the tissue of the organ or the organ itself in any manner sufficient for the specific desired application. In alternative embodiments, the probes may reside on the surface of a body. The probe may be affixed to the surface of the body such that it resides next to and in close proximity to the skin of the body. The probe may be affixed to the surface of the body in any means sufficient for a specific desired application. Such means may include bands, wrappings, stickers, tape, adhesive materials/solutions, and the like. The probe may be affixed to any part or portion of the body such as limbs, hands, fingers, core, torso, head, neck, etc.

In further alternative embodiments, the probe may be a handheld device. The handheld probe device may comprise any of the light sources or photodetectors described above and in any combination as described above. Preferred embodiments of the handheld probe comprise a wide field camera photodetector (e.g., a CCD photodetector). In embodiments of the handheld probe comprising an array of photodetectors, the handheld probe may take other body measurements such as blood pressure or detect and monitor the development of pressure ulcers.

In embodiments, the probes may be positioned on any part of the tissue, and/or inserted into the tissue, and/or held away from, but focused onto the tissue. For example, in a specific embodiment, a probe is positioned to measure compliance and vascular resistance in hepatic tissue. The probe may be positioned on any part of the hepatic parenchyma to monitor changes in tissue properties over time. In this embodiment, only one probe is needed, however additional probes may be used to monitor other locations and study heterogeneity in mechanical properties. In embodiments, the probe may be affixed to the tissue using any sufficient means. In alternative embodiments, the probe may be located on a handheld apparatus and positioned over and/or focused on the tissue to be examined. In further embodiments, the probe may be placed on or into the tissue such that it may remain for a desired measure of time until monitoring is no longer needed or it is desirable to remove it.

Embodiments may comprise a main unit. In some embodiments, the main unit may drive the light sources, collect the data from the detectors, and/or process and display the collected data as measurements. The main unit may be a component of or may be separate from the probe. In embodiments where the main unit is separate from the probe, the main unit may connect wirelessly with the probe; in further alternative embodiments, the probe may dock and/or mate with the main unit such that the probe and the main unit may interact. Without limitation, examples of docking and/or mating may include use of wire interface (e.g. USB, Ethernet, serial interface, and the like). In alternative embodiments, the main unit may be tethered to the probe such that it is connected to the probe yet at a distance away from the tissue or body to be examined. The main unit may comprise one or more circuit boards. The main unit may comprise one or more microcontrollers. The main unit may communicate wirelessly with a remote relay station and/or a remote personal computer such as a computer, smart-phone, tablet, etc. In embodiments wherein the main unit is a component of the probe, the main unit may be encapsulated in the same manner as any other component of the probe may be encapsulated. In embodiments, wherein the main unit is distinct from the probe, the main unit may be encapsulated or may not be encapsulated.

Embodiments may comprise a transmitter and/or a receiver for wireless communication. The transmitter and/or receiver may be a component(s) of the probe and/or the main unit, either individually or in conjunction with each other. In embodiments, the system may communicate with any mobile device (i.e., phone, smart watch, etc.) directly or through a relay unit. Without limitation, the transmitter and receiver may comprise communication means such as radio waves, infrared signals, audio, and electro-magnetic waves, for example active RF (e.g., WiFi, WiFi 802.11, Bluetooth®, 3G, and the like), RFID (e.g., Near Field Communication (NFC), both active and passive RFID as well as low and high frequency and the like), an infrared or optical link (LED's and the like), and/or any other suitable data transfer type, device, or method.

Embodiments may comprise a power source. The power source may be any type of battery (primary or secondary) capable of providing power to drive the main unit and the probe for the desired data collection duration. Specific examples of batteries include but are not limited to lithium ion batteries, lithium/carbon monoflouride (Li/CFx), lithium/silver vanadium oxide (SVO), lithium iodine, alkaline batteries, nickel-zinc batteries, or other battery technologies. The power can also be supplied through an alternative source or sources including inductive power coupling, optical, ultrasonic/ultrasound, motion, or a scavenged energy source (heat, vibration, ambient light, chemical, or acoustic). Depending on the requirements of the application, a combination of these methods may be used such as lithium ion batteries charged via inductive power coupling.

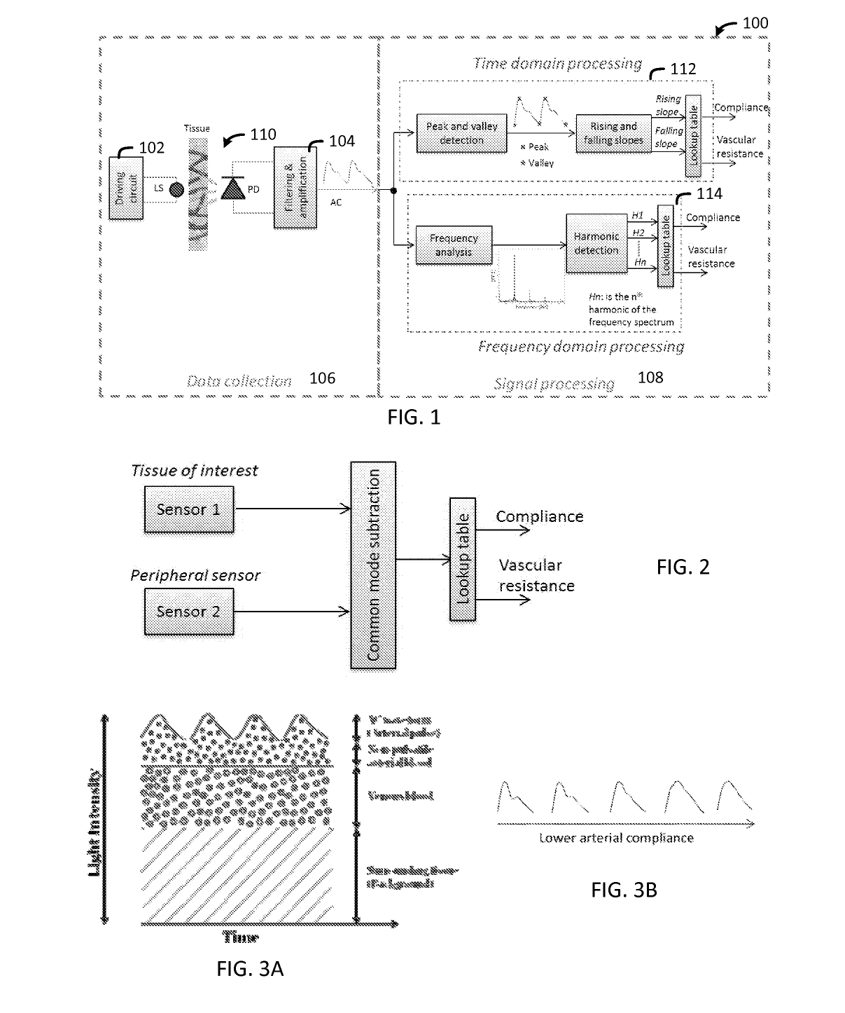

Embodiments may comprise a method 100 for deducing tissue mechanical properties as shown in FIG. 1. The method comprises illuminating tissue with different wavelengths of light. A photodetector (PD) collects the reflected light data after the light has propagated through the tissue. The reflected light data collected by the detector (PD) comprises a pulsatile alternating current component. The waveform of the AC current can then be studied by measuring the frequency, time domain, or the joint time-frequency domain of the waveform. The frequency and time domain of the waveform may provide diagnostic information that may be used to evaluate the mechanical properties of tissue.

The probe 102 and the main unit 104 are represented by the electronics on the left side (Data collection 106) whereas the data analysis of the collected signal is represented on the right side (Signal processing 108). The probe 102 is preferably non-invasive. For example, the probe 102 can be affixed to or in close proximity to a surface of the tissue. Moreover, the probe 102 can be integrated into, directly connected, tethered or wirelessly connected to the main unit 104. Other possible characteristics and configurations of the probes 102 and main unit 104 were previously described.

The driving circuit 102 includes one or more light sources (LS) that illuminate the tissue with a light having one or more wavelengths (e.g., 735, 805, 940 nm, etc.). The main unit 102 provides common filtering and application of the reflectance signal 110 received by the one or more photodetectors (PD). The reflectance signal 110 includes an AC component (AC). The signal processing 108 can be performed using one or more processors within the main unit 104 or remotely located with respect to the main unit 104. In this example, the signal processing 108 includes time domain processing 112 and frequency domain processing 114. The time domain processing 112 detects peaks and valleys of the AC component (AC), detects rising and falling slopes of the AC component (AC) and uses a lookup table to determine compliance and vascular resistance. The frequency domain processing 114 performs a frequency analysis, detects harmonics and uses a lookup table to determine compliance and vascular resistance.

In embodiments, two probes may be used in distinct areas and/or tissues to measure peripheral readings in addition to the readings for the area/tissue of interest as shown in flowchart for the process in FIG. 2. This is done so that the readings taken from the tissue of interest may be compared to the peripheral reading to account for any systemic effects on the pulse.

In a specific embodiment and as an example, a custom bench-top PPG system was used to collect the data. In summary, the system uses three time multiplexed light emitting diodes (LEDs) emitting light at different wavelengths in the red to near infrared spectral region (735, 805, and 940 nm). The diffuse reflectance is collected using a single photodetector. The collected signal on each wavelength is filtered and split into two channels: (1) an AC (alternative current) channel that records the amplified photoplethysmogram; and (2) a DC (direct current) channel that encompasses all slow varying signals. The AC and DC channels may both be needed when performing perfusion and oxygenation measurements. However, in these embodiments, since the focus is on the PPG waveform, only the AC channel was used in the processing. FIG. 3A shows a schematic of the collected signal before filtration and separation of the AC and DC components. FIG. 3B shows changes in the shape of the pulse with arterial compliance.

To understand the reasoning behind the signal processing of the method, the following will describe a simplified origin of the pulse. When blood flows into a capillary bed, it encounters a resistance due to the size and distribution of the vessels, the compliance of the vessels and surrounding tissue, and many other factors that depend on the mechanical structure of the tissue and vasculature. In addition, it also encounters a back flow due to a reflected wave generated at a resistance mismatch point such as the peripheral vessels or the aortic valve. These properties give the pulse its shape which is different from the shape of the cardiac output (blood flow out of the heart). This concept can also be reduced to the level of hepatic blood flow. To simplify the description, the liver can be thought of as an RC electric circuit where the resistance is the vascular resistance to the flow and the capacitance is the compliance of the hepatic circulation. The current represents the blood flow while the electric potential (voltage) mimics the blood pressure that controls the blood volume in the capillary bed. When the resistance increases, the time constant (RC), that represents the temporal response of the system, also increases. This is the case of a downstream vascular blockage or narrowing. Note that when the narrowing or blockage takes place upstream from the measurement site, the resistance is unaffected and the time constant is not expected to change. Similarly, when the capacitance decreases, the time constant decreases as well which leads to a decreased time constant. This describes the case of decreased compliance or stiffening of the tissue that can be due either to hepatic edema, which is very common after transplant, or fibrosis.

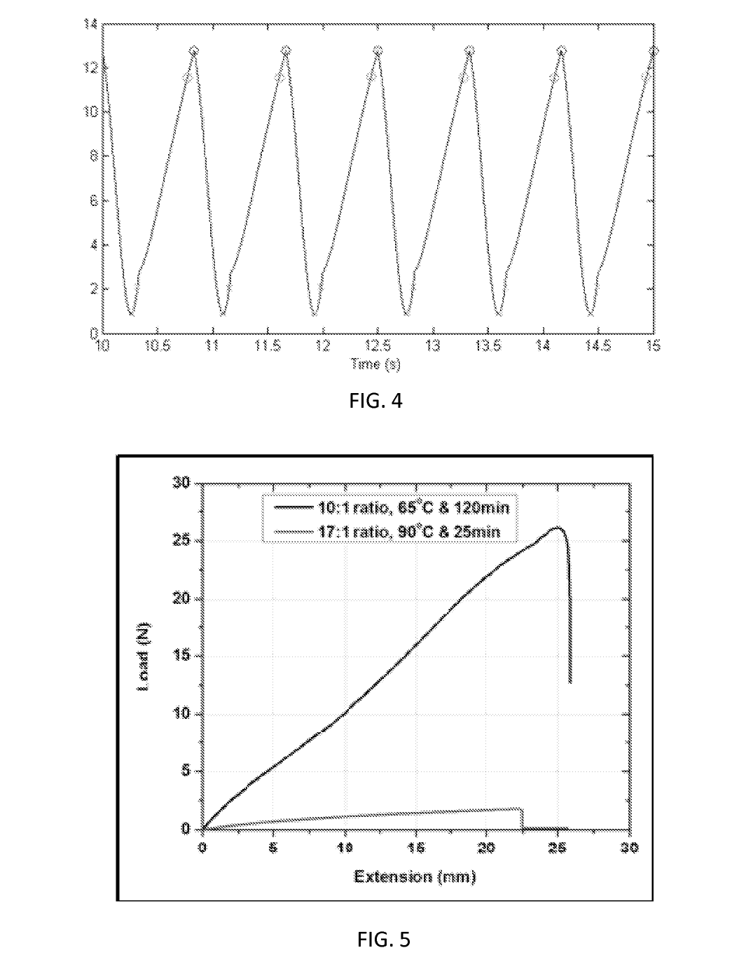

To obtain a quantifiable measure of this time constant, in embodiments, the rise time in the PPG pulse is measured which corresponds to the decrease in tissue blood volume that happens during diastole. The rise time is defined herein as the time between the foot of the pulse and the peak. To avoid any errors due to noise causing fluctuations during these periods, the time between the point that is 10% larger than the valley and the point that is 10% lower than the peak was used as shown in FIG. 4.

In embodiments, the tissue mechanical properties monitoring system may also be used to monitor additional metrics. For example, the tissue mechanical properties monitoring system may monitor blood pressure, blood perfusion, heart rate, and the like. This information may be used by itself or in conjunction with any other diagnostic information to produce insight for healthcare providers or information relevant to the state of the tissue that may be used directly by the patient themselves.

In embodiments, a calibration model may be used to calculate tissue compliance and vascular resistance. In embodiments, the tissue mechanical properties monitoring system may comprise a display or monitor such that information about tissue compliance, vascular resistance, heart rate, and/or respiratory rate, etc. may be displayed in such a manner to easily convey the details of this data to a monitoring physician or other type of healthcare provider.

Various non-limiting examples of embodiments of the present invention will now be described. The proposed concept was tested in a series of in vitro phantom studies. Polydimethylsiloxane (PDMS) phantoms were fabricated with different curing parameters to adjust their mechanical properties. PDMS was mixed with various optical absorbers (blue food coloring and black India ink) and scatterers (100 nm and 0.5-1 μm Aluminum Oxide powder) to mimic the optical properties of hepatic tissue in the 630-1,000 nm wavelength range. The phantoms mimic the structures of the portal vein (PV), the main blood and nutrients supplier to the liver. The curing parameters that control the PDMS mechanical properties include the curing temperature, curing time, and the concentration of the curing agent.

For the purpose of this study, three different sets of phantoms were fabricated. The curing time and temperature were kept at 24 hours and 60° C. for all three phantoms while the PDMS to curing agent volumetric ratio was changed between 30:1, 40:1, and 45:1 v/v which yielded a Young's modulus (YM) of 11.7, 15, and 61 KPa respectively. All YM measurements were obtained from stress-strain curve measurements obtained by an Instron® 3345 (Instron, MA, USA). The calculations were made using an automated program developed in MATLAB. Note that compliance is inversely proportional to the fourth root of the Young's modulus (C∝1/E) and a larger YM indicates a less compliant material. All the reported measurements are for the PDMS with the optical absorbing and scattering agents which has a higher YM in comparison to clear PDMS. Similarly, to avoid handling blood, an optical mixture of various optical dyes was used to mimic the optical properties of oxygenated hemoglobin. FIG. 5 illustrates the mechanical properties of two different PDMS phantoms with different curing parameters. These phantoms were used to test the proposed concept.

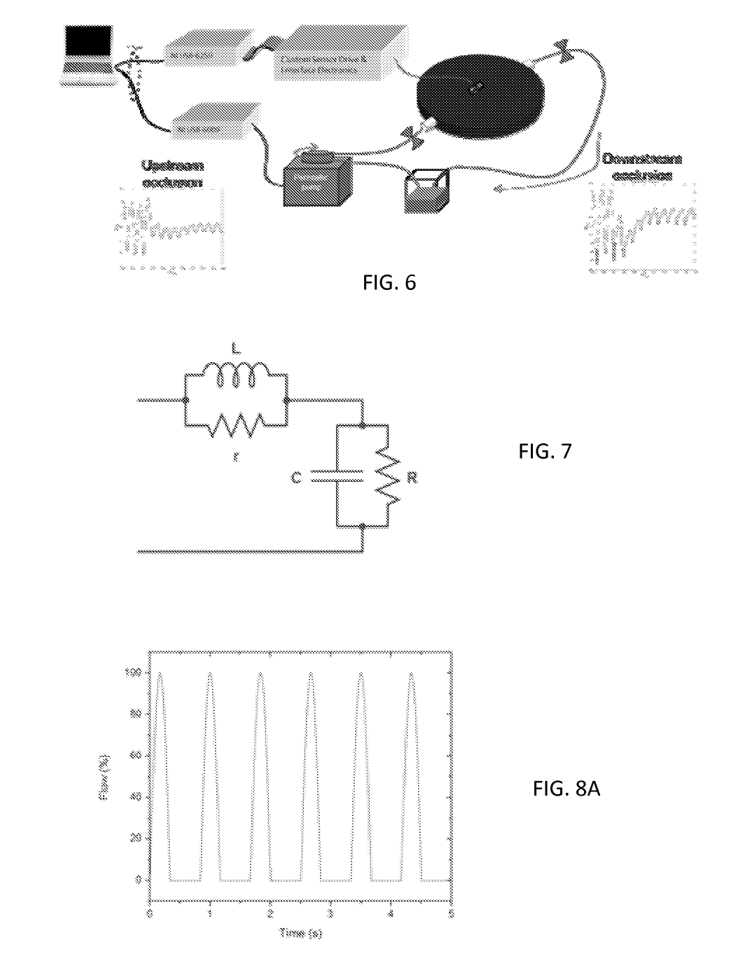

The phantoms described above were connected to a fluidic circuit to mimic the pulsatile blood flow. A peristaltic pump controlled via a virtual instrument (VI) was used to control the pulsatile flow. The phantoms were perfused with the dye mixture and c-clamps were placed on the tubing on either side of the phantom to occlude flow when needed. The PPG probe was placed on top of the phantom and held in place with a mechanical arm. FIG. 6 shows a schematic of the system. The insets show data collected from the phantom experiments during a downstream and an upstream occlusion. Note the change in the waveform when a downstream occlusion is performed.

To study the expected performance of the system theoretically over a wider range of physiologic conditions, a four element Windkessel model was developed. The inductive element represents the inertia of blood flow. FIG. 7 shows a schematic of the four element Windkessel model. Note that this model is not meant to be an accurate representation of hepatic circulation but more of a general model to mimic physiologic signals and highlight the expected changes in the pulse with various parameters. The blood flow was modeled by equation I:

$$i ( t ) = \{ \begin{matrix}{I}_{0} \cdot sin ( π \cdot mod ( t , 60 \text{/} HR ) \cdot \frac{HR}{60 \cdot {t}_{s}} , & mod ( t , 60 \text{/} HR ) \leq \frac{HR}{60 \cdot {t}_{s}} \\ 0 , & otherwise\end{matrix} \qquad ( I )$$

Where I₀ represents the peak blood flow and was set to 500 mL/s. HR is the heart rate in beats per minute (bpm) and tₛ is the ratio of the systole time divided by the cardiac cycle time. HR and tₛ were set to 72 bpm and 0.4 respectively. “mod” refers to the modulo operation.

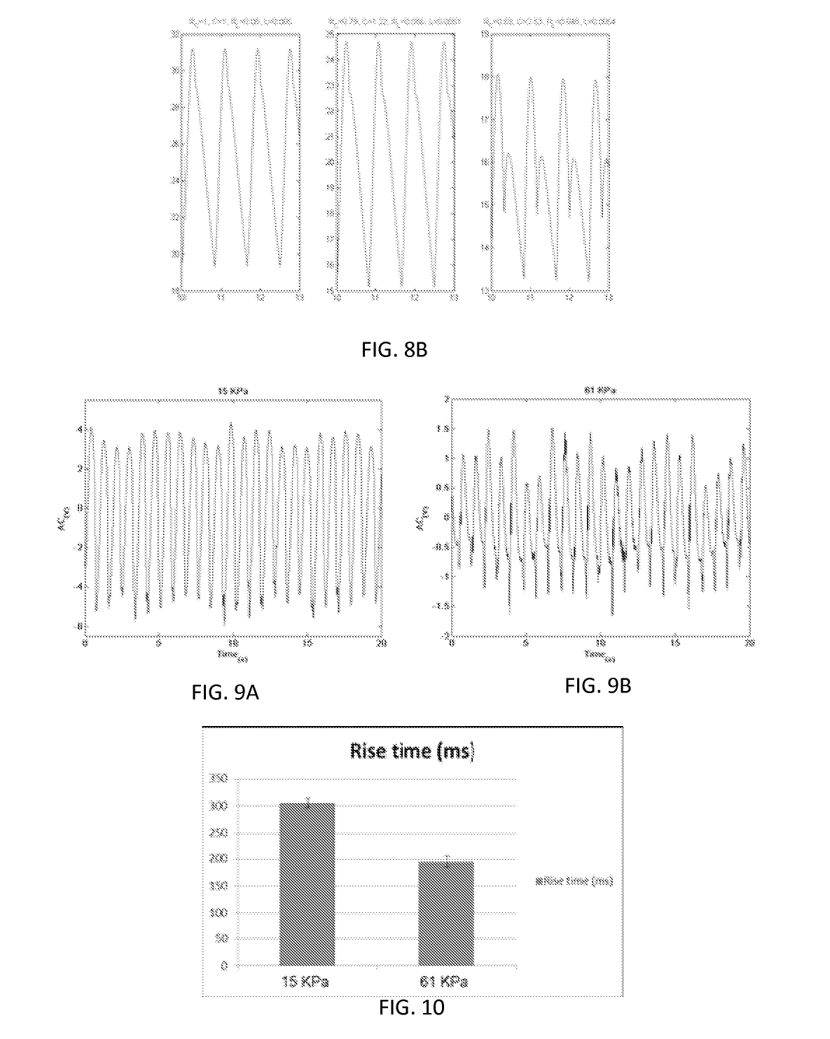

As discussed earlier, the modeled pulse (blood pressure and/or volume) mimics the change in optical absorption. To get the changes in optical intensity which is measured by the PPG sensor, the approximation shown in equation II was used. This approximation can be used since only the waveform of the pulse, not the amplitude of the pulse is of interest. The blood flow signal and three different pressure waveforms obtained by the Windkessel model are shown in FIGS. 8A and 8B.

I∝Pₘₐₓ−P (II)

The different phantoms were placed in the flow circuit described above and the benchtop PPG system was used to measure the pulsatile signal. FIGS. 9A and 9B show two waveforms measured from two different phantoms with different mechanical properties (soft (15 KPa) and stiff (61 Kpa), respectively).

The rise time was measured from one minute of continuous data for each phantom. This was repeated three times for each phantom and the average and standard deviation were calculated accordingly. As expected, the rise time decreased with increased Young's modulus (decreased compliance) as shown in the bar charts of FIG. 10. Note that the rise time is also affected by the changes in the flow circuit (tubing material, tubing dimensions, pump, connectors, etc.).

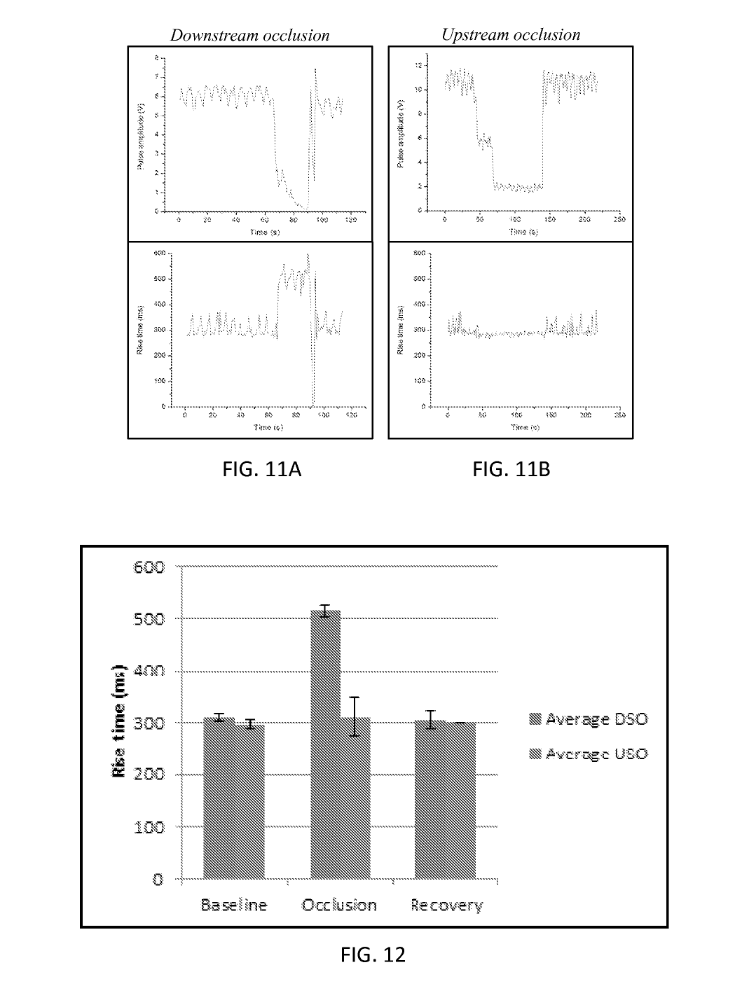

For each of the phantoms, after collecting baseline data for 30-60 s, flow was occluded using a c-clamp either upstream or downstream from the phantom. For the first occlusion, the clamp was tightened until a visual decrease in pulse amplitude can be seen. The same number of turns on the c-clamp was used for every occlusion afterwards. During both, upstream and downstream occlusions, the amplitude of the PPG signal decreased indicating a decrease in the pulsatile flow. However, in the case of downstream occlusions, an increase in the PPG rise time was recorded which is expected due to the increase in the resistance. This was not seen during upstream occlusions. FIG. 11A shows an example of a downstream occlusion and FIG. 11B shows an example of an upstream occlusion. In both cases, the amplitude of the pulse (top line) decreases indicating a drop in flow level. The rise time (bottom line) increased only in the case of downstream occlusions. The rise time in the recovery period, after the occlusion was released, was the same as the baseline value. This experiment was repeated three times for every type of occlusions. The bar graphs in FIG. 12 show the average and standard deviation of all runs. The red or left bars correspond to the average change in rise time during downstream occlusions. The blue or right bars correspond to the average change in rise time during upstream occlusions. The error bars correspond to +/− on standard deviation.

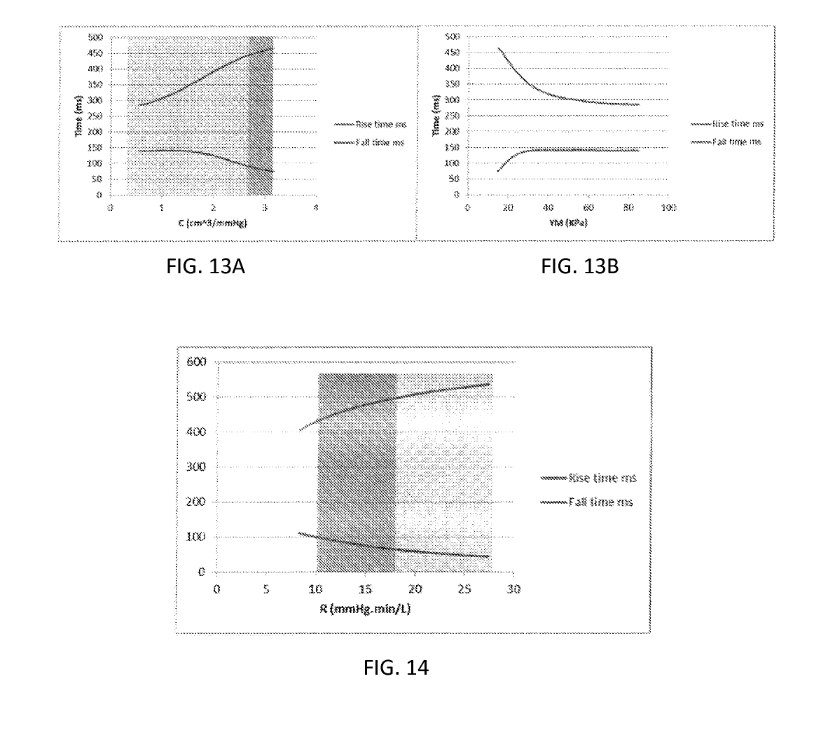

To test the proposed concept over a wider range of parameters, the Windkessel model described earlier was used. The compliance was changed between 0.55 and 3.15 cm3/mmHg which correspond to Young's modulus of 60.6 to 10.6 KPa respectively. Note that the relationship between compliance and Young's modulus is not linear. The compliance is proportional to the inverse of Young's modulus (C∝1/E). Similar to the in vitro data, the rise time increased for higher compliance levels while the fall time decreased (FIGS. 13A and 13B). In FIG. 13A, the darker shaded area indicates the range for normal tissue while the lighter shaded area corresponds to fibrotic tissue at different stages. FIG. 13B shows the data after conversion of the compliance values to YM.

To mimic vascular occlusions, the case of increased resistance was modeled. The normal physiologic range of systemic vascular resistance is typically in the range of 11.25 to 18 mmHg·min/L (1,170+/−270 dynes-sec/cm5). Resistance changes between 8.3 and 27.3 mmHg·min/L were modeled which covers the normal range and the elevated resistance range as shown in FIG. 14. The darker shaded area indicates the normal range while the lighter shaded area corresponds to increased vascular resistance.

As described above, a method for determining mechanical properties of a tissue has been disclosed. A probe is provided that is affixed to or in close proximity to a surface of the tissue. The probe includes one or more light sources and one or more photodetectors. The light emitted by the one or more light source may include three or more wavelengths of light (e.g., a first wavelength of approximately 735 nm, a second wavelength of approximately 805 nm, and a third wavelength of approximately 940 nm).

One or more processors communicably coupled to the probe and a data output device are also provided. The tissue is illuminated using the one or more light sources, and a reflectance signal is detected using the one or more photodetectors. The reflectance signal includes an AC component. The mechanical properties for the tissue are determined based on the reflectance signal using the one or more processors. The mechanical properties for the tissue, such as the fibrosis, cirrosis, wound healing, tissue burn monitoring, edema, etc., are then provided to the output device. An additional probe communicably coupled to the one or more processors and configured to measure peripheral readings for the tissue may be provided.

The step of determining the mechanical properties for the tissue based on the reflectance signal using the one or more processors comprises determining a compliance and a vascular resistance in both a time domain and a frequency domain. The method may also include the step of time multiplexing or frequency multiplexing the one or more photodetectors to collect the reflectance signal at each of the three or more wavelengths of light using frequency modulation, time division multiplexing or a combination thereof. Similarly, the method may include the step of modulating the one or more light sources such that the light is at a different frequency than an ambient light.

Note that embodiments of the present invention can be used for “Arterial and Venous Oxygenation Method and Apparatus” as disclosed in U.S. patent application Ser. No. 14/608,145 filed concurrently herewith and provisional patent application Ser. No. 61/932,575 filed on Jan. 28, 2014, both having that title and incorporated by reference in their entirety.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or rives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Although the present invention and its advantages have been described in detail, it should be understood that various changes, substitutions and alterations may be made herein without departing from the spirit and scope of the invention as defined by the appended claims. The scope of this disclosure is not limited to the example embodiments described or illustrated herein. Moreover, although this disclosure describes and illustrates respective embodiments herein as including particular components, elements, functions, operations, or steps, any of these embodiments may include any combination or permutation of any of the components, elements, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative.

What is claimed is:

1. A tissue mechanical property monitoring system comprising:
a probe comprising a light source and a photodetector;
a main unit comprising a microcontroller and a communications interface with the probe;
wherein the probe is hermetically sealed and is capable of being implanted onto tissue;
wherein the photodetector is configured to collect a reflectance data from the a light emitted by the light source that illuminates the tissue; and
wherein the microprocessor processes the reflectance data into a tissue mechanical property data for the tissue.

2. The system as recited in claim 1, wherein the probe is hermetically sealed and is configured to be affixed to or in close proximity to a surface of the tissue.

3. The system as recited in claim 1, wherein the probe is integrated into, directly connected, tethered or wirelessly connected to the main unit.

4. The system as recited in claim 1, further comprising an additional probe configured to measure peripheral readings for the tissue.

5. The system as recited in claim 1, wherein the reflectance data comprises a reflectance signal having an AC component.

6. The system as recited in claim 1, wherein the tissue mechanical property data comprises one or more of fibrosis, cirrosis, wound healing, tissue burn monitoring and edema.

7. The system as recited in claim 1, wherein the microcontroller determines a compliance and a vascular resistance in both a time domain and a frequency domain.

8. The system as recited in claim 1, wherein the light emitted by the light source comprises three or more wavelengths of light.

9. The system as recited in claim 8, wherein the three or more wavelengths of light comprise a first wavelength of approximately 735 nm, a second wavelength of approximately 805 nm, and a third wavelength of approximately 940 nm.

10. The system as recited in claim 9, wherein the photodetector is time multiplexed or frequency multiplexed to collect the reflectance data at each of the three or more wavelengths of light using frequency modulation, time division multiplexing or a combination thereof.

11. The system as recited in claim 1, wherein the light source modulated the light such that the light is at a different frequency than an ambient light.

12. A method for monitoring mechanical properties of a tissue, comprising the steps of:
providing a probe affixed to or in close proximity to a surface of the tissue, wherein the probe comprises one or more light sources and one or more photodetectors;
providing one or more processors communicably coupled to the probe and a data output device;
illuminating the tissue using the one or more light sources;
detecting a reflectance signal using the one or more photodetectors;
determining the mechanical properties for the tissue based on the reflectance signal using the one or more processors; and
providing the mechanical properties for the tissue to the output device.

13. The method as recited in claim 12, further comprising an additional probe communicably coupled to the one or more processors and configured to measure peripheral readings for the tissue.

14. The method as recited in claim 12, wherein the reflectance signal comprises an AC component.

15. The method as recited in claim 12, wherein the tissue mechanical property data comprises one or more of fibrosis, cirrosis, wound healing, tissue burn monitoring and edema.

16. The method as recited in claim 12, wherein the step of determining the mechanical properties for the tissue based on the reflectance signal using the one or more processors comprises determining a compliance and a vascular resistance in both a time domain and a frequency domain.

17. The method as recited in claim 12, wherein the light emitted by the one or more light source comprises three or more wavelengths of light.

18. The method as recited in claim 17, wherein the three or more wavelengths of light comprise a first wavelength of approximately 735 nm, a second wavelength of approximately 805 nm, and a third wavelength of approximately 940 nm.

19. The method as recited in claim 18, further comprising the step of time multiplexing or frequency multiplexing the one or more photodetectors to collect the reflectance signal at each of the three or more wavelengths of light using frequency modulation, time division multiplexing or a combination thereof.

20. The method as recited in claim 12, further comprising the step of modulating the one or more light sources such that the light is at a different frequency than an ambient light.
